(19) United States
(12) Patent Application Publication
(10) Pub. No.: US 2011/0137413 A1
(43) Pub. Date: Jun. 9, 2011

(54) BREAST IMPLANT

(75) Inventor: Peter Osypka, Grenzach-Wyhlen (DE)

(73) Assignee: Peter Osypka Stiftung Stiftung des burgerlichen Rechts, Grenzach-Wyhlen (DE)

(21) Appl. No.: 12/959,948

(22) Filed: Dec. 3, 2010

(30) Foreign Application Priority Data
Dec. 4, 2009 (DE) 202009016559.0

(51) Int. Cl. A61F 2/12

(52) U.S. Cl. 623/8

(57) ABSTRACT

A breast implant (1) having a flexible, but impermeable casing (2) and with an electrically conductive filling (3) is provided with an internal electrode (4) and an external electrode (5), with the electrodes being connected to a resistance or impedance measurement device integrated in a transponder (6). In case of perforation of the casing (2), if filling (3) escapes, then the electrical contact between the electrodes (4) and (5) is completed, and a drop in resistance or impedance is registered by the measurement device. In suspicious cases, the implant could be inspected of the measured resistance or impedance values with an external transmitter-receiver unit (13 or 14) and dangerous damage to the breast implant could be determined.

### CROSS-REFERENCE TO RELATED APPLICATIONS

This application claims the benefit of German Patent Application No. 20 2009 016 559.0, filed Dec. 4, 2009, which is incorporated herein by reference as if fully set forth.

### BACKGROUND

The invention relates to a breast implant, in particular, for women, with a flexible, but impermeable casing and with a filling located in this casing.

Such breast implants have been known and in use for a long time. Despite careful manufacture and implantation, however, the casing of the implant can become permeable at least in some locations, can start to tear, or can have some other damage through which the filling can escape, for example, due to an accident.

With the escape of the filling, medical complications can materialize. In addition to negative aesthetic effects, such as deformations and hardening of the previous filling in the breast, which often make additional surgeries necessary, serious and harmful effects to health can occur.

In particular, the tissue surrounding the implant could be negatively affected by such an escape of the filling.

Examinations for corresponding complaints, for example, by ultrasound, X-ray, or MRT, are complicated and expensive.

### SUMMARY

Therefore, there exists the objective of creating an implant of the type defined above in which damage resulting in the escape of filling can be determined as easily as possible.

To meet this objective, the implant defined above is characterized in that the implant has, in its interior, at least one electrical pole or one electrode and on its outside at least one additional electrical pole or one electrode, that the two poles or electrodes are connected to an electrical resistance or impedance measurement device, and that the filling of the implant is electrically conductive.

Because the two electrodes are separated from each other electrically by the casing of the implant, in the normal case there exists a very high impedance or a very high resistance. If damage occurs on the casing of the implant leading to a discharge of a portion of the electrically conductive filling, a significantly smaller resistance is produced between the two poles or electrodes across the filling remaining in the implant, the opening of the casing produced by the damage, and the discharged portion of the filling, as well as the body tissue and/or the body fluid, wherein this lower resistance can then be used as a measurement signal for the defective or damaged state of the implant. The indication of the drop in resistance or impedance could now be realized to the outside by an acoustic signal or a vibrating alarm.

The electrically conductive filling could be, in a known method, a saline solution or a gel filling with additives making it conductive. Such additives could be, for example, carbon or metal powder.

For simplifying the inspection of the breast implant and the transmission of the resistance or impedance values necessary for this inspection, it is especially useful if the two poles or electrodes are connected to a transponder that contains the measurement device and that is constructed for transmitting the measurement values to an external receiver.

One possibility for the construction of the implant could provide that the transponder contains, as the measurement device, a resistor measurement bridge. Thus, the installation space required for the transponder and the measurement device could be kept as small as possible in or on the implant.

In order to preserve the shape of the inserted implant adapted to the natural anatomy despite the attached poles or electrodes, the transponder, and the measurement device, it is especially useful if the electrical pole or electrode located in the interior of the implant is arranged or fastened on the inner side to the rear wall or back side of the implant in the position of use.

In the same way, it is then preferred if the outer pole or electrode is arranged or fastened on the outer side to the rear wall or back side of the implant in the position of use. On one hand, this arrangement does not visibly affect the anatomy and, on the other hand, the natural flexibility of the implant is not at all or only slightly negatively affected.

The discharge of electrically conductive filling could be detected by a reduction of the measurable resistance or the measurable impedance if the two poles or electrodes were previously separated electrically from each other by the wall of the implant and/or were also isolated relative to each other.

It is especially useful if the position or wall holding the outer electrode or the outer pole has a recess for the pole or the electrode and thus the pole or the electrode is countersunk relative to the surface of the outer side in the position of use at least partially or such that the outer side of the pole or the electrode is flush with the outer side of the wall holding it.

Thus, the outer electrode projects only slightly or not at all from the surface of the implant and undesired changes due to the outer electrode with respect to its defining shape can be avoided.

One advantageous construction of the invention and thus useful for the application of the breast implant is if an opening is provided on the back wall of the implant for the insertion of the implant filling and a closure for this opening is provided and the outer pole or the electrode, the transponder, and the inner pole or the electrode are arranged on or in the closure.

With this compact construction and the integration of the two electrodes, the transponder, and the measurement device into the closure, undesired effects on the shape of the implant could be avoided.

In order to be able to make a faster detection of escaping filling fluid or filling gel from the implant, lines or electrically conductive arms that extend from the pole or electrode and that are arranged at least on the rear outer side of the back wall of the implant in the position of use or extend beyond the front side of the implant could be connected to the outer pole or electrode.

This arrangement produces, accordingly, shorter paths for the escaping filling material to come in contact with parts of the outer electrode and thus producing a drop in resistance or impedance. The closer the outer electrode or parts of this electrode are located to the discharge location, the faster the discharge of filling and thus the damage can be determined by the drop in the measured resistance or impedance.

Another construction of the implant provides that the additional, electrically conductive arms are constructed as elastically deformable, electrically conductive bands.

A breast implant constructed in this way allows all of the elements required for the implant monitoring to be installed, so that the outer shape of the implant differs not at all or only slightly from such an implant without this device. Consequently, such bands do not affect the shape of the implant, but can cover large areas of its surface and can thus reduce the distances between any discharge and failure position and these bands and can thus reduce the time period between discharge of filling and detection by the monitoring device.

### BRIEF DESCRIPTION OF THE DRAWINGS

Below, embodiments of the invention are described in detail with reference to drawings. Shown in partially schematized diagram are:

FIG. 1 is a front view of a breast implant according to the invention, here shown transparent, with an impermeable casing, an electrically conductive filling, and the partially visible poles or electrodes,

FIG. 2 is a side view of the breast implant according to FIG. 1,

FIG. 3 is a diagram corresponding to FIG. 2 supplemented by the schematic figure of a co-implanted transponder that contains a resistance or impedance measurement device,

FIG. 4 is an enlarged side view of the arrangement of the poles or electrodes on the rear wall or back side of the implant,

FIG. 5 is a diagram of a modified embodiment corresponding to FIG. 1 in which electrically conductive arms or bands run in the vertical and horizontal direction across the back side of the implant,

FIG. 6 is a diagram corresponding to FIG. 5 of a modified embodiment in which an electrically conductive band surrounds the rear face of the implant,

FIG. 7 is a diagram corresponding to FIG. 1 of a modified embodiment in which the electrically conductive bands are guided beyond the front side of the implant,

FIG. 8 is a diagram corresponding to FIG. 7 in side view,

FIG. 9 is an implant according to the invention in the position of use and a transmitter-receiver unit provided for interaction with a passive transponder,

FIG. 10 is a diagram corresponding to FIG. 1 in the construction with an active transponder.

### DETAILED DESCRIPTION OF THE PREFERRED EMBODIMENT

A breast implant designated overall with 1 with a flexible, but impermeable casing 2 and with an electrically conductive filling 3 located in this casing carries, in its interior, at least one electrical pole or one electrode 4, called “internal electrode” below, and, on its outside, another electrical pole or electrode 5, called “external electrode” below. The two electrodes 4 and 5 are connected to an electrical resistance or impedance measurement device that is not shown explicitly and that is called “measurement device” below.

Here, the measurement device could be integrated in a transponder 6 that could be located, as in FIG. 3, outside of the implant or, as in FIGS. 2, 4, and 8, within the implant.

In FIG. 2, the position of the two electrodes 4 and 5 in relation to the transponder 6 can be seen. The internal electrode 4 is fastened on the inner side facing the implant and the external electrode 5 is fastened to the outer side of the transponder 6 facing away from the implant. The transponder itself is set in the back wall 7 of the implant. The electrodes 4 and 5 are separated electrically from each other by the casing 2 of the implant and by the transponder 6.

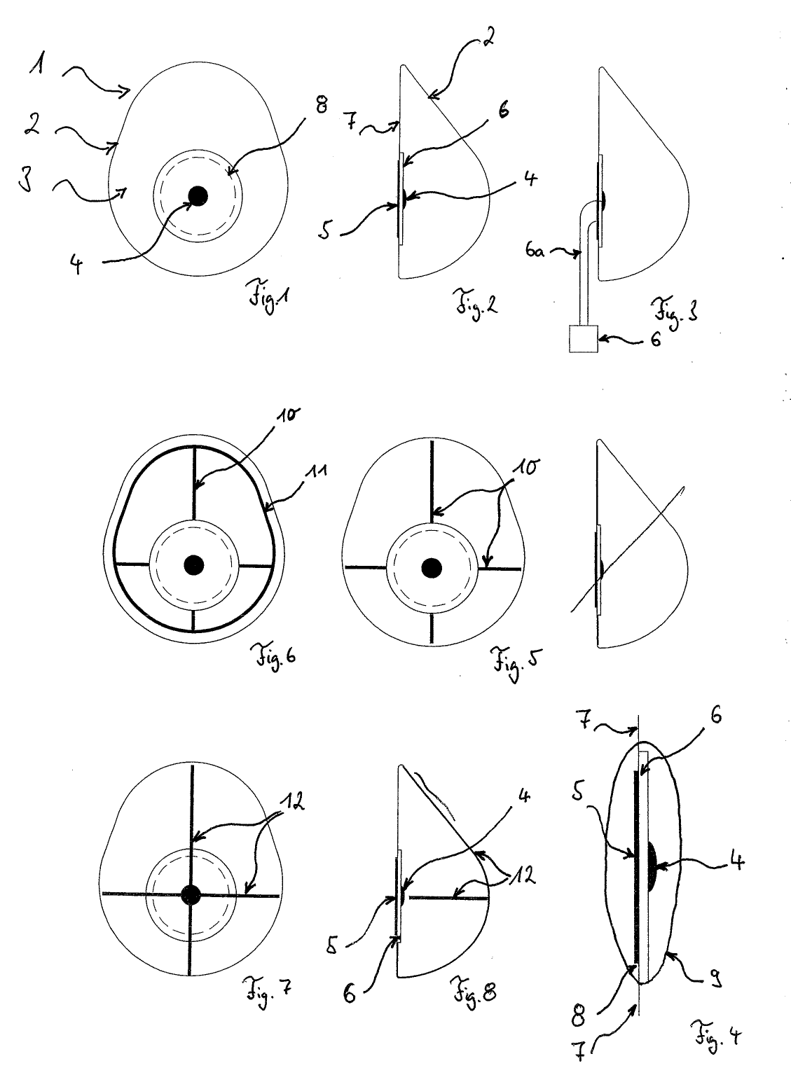

As FIGS. 1 to 8 show, the implant can have an opening 8 for inserting the implant filling 3 on its back side 7. This opening 8 could be sealed by a closure 9 shown enlarged in FIG. 4.

The closure 9 includes the transponder 6 on which the internal electrode 4 is attached on the inner side of the implant and the external electrode 5 on the outer side of the implant.

After insertion of the implant filling, the closure 9 is placed and bonded with the back side 7 of the implant and seals the opening 8 of the implant.

FIG. 5 shows another element of the breast implant. For increasing the contact face of the external electrode 5, additional, electrically conductive arms or bands 10 are provided that run in the horizontal and vertical directions on the back wall 7 of the implant.

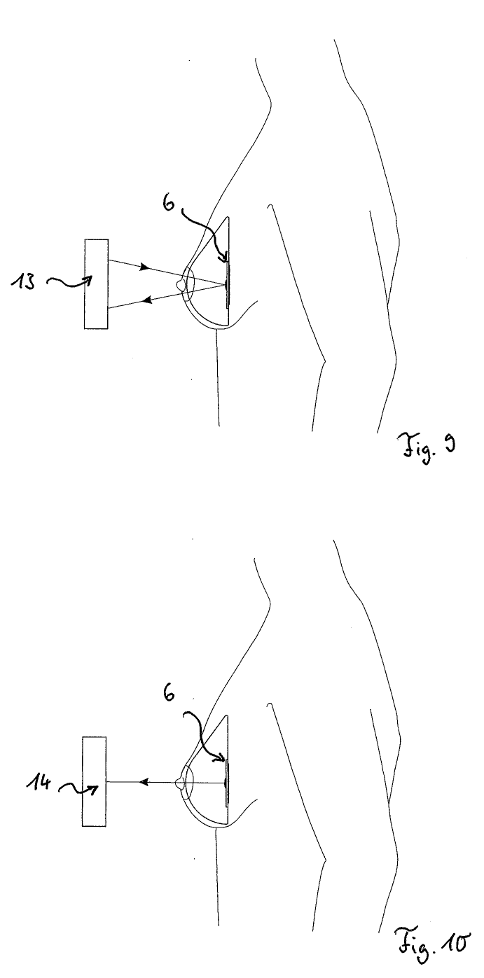

FIG. 6 shows another construction of the external electrode. An electrically conductive band 11 encompassing the area of the back wall 7 attaches to the electrically conductive arms 10.

The contact face of the external electrode 5 can be further increased, as described in FIGS. 7 and 8, by guiding the electrically conductive arms 12 up to the front side of the implant.

In the case of a perforation of the casing 2 of the implant, if electrically conductive filling 3 escapes to the outside, then the electrical contact between the internal electrode 4 and the external electrode 5 closes directly via the filling and/or via body tissue or fluid. Here, the measurement device registers a drop in resistance or impedance.

According to the embodiments from FIGS. 5 to 8, the additional, electrically conductive arms 10, 11, and 12 allow an earlier contacting of the two electrodes and thus an earlier detection of the discharge of the implant filling. Through use of the transponder 6, the measured impedance or resistance value can be transmitted to an external receiver.

FIG. 9 shows such a transmitter-receiver unit 13 and the implant in the position of use. The transponder 6 is here a passive transponder, thus it does not have its own power supply, but instead is powered by the electromagnetic field of the transmitter-receiver unit. As soon as it is excited, it can transmit the measured impedance or resistance measured values to the external transmitter-receiver 13.

FIG. 10 shows another construction of the implant according to the invention with an active transponder 6 that has its own power supply not shown in detail here, for example, a battery. Consequently, the transponder could also transmit data and measurement values to the external receiver 14 without excitation or power from the electromagnetic field of a transmitter-receiver unit and could optionally indicate damage to the casing 2 of the implant in the case of discharge of filling 3.

A breast implant 1 according to the invention with a flexible, but impermeable casing 2 and with an electrically conductive filling 3 has an internal electrode 4 and an external electrode 5, with these electrodes being connected to a resistance or impedance measurement device integrated in a transponder 6.

In the case of a perforation of the casing 2, if filling 3 is discharged, the electrical contact between the electrodes 4 and 5 is closed and a drop in resistance or impedance is registered by the measurement device. In suspicious cases, the implant could be inspected by the measured resistance or impedance values with an external transmitter-receiver unit 13 or 14 and dangerous damage to the breast implant could be determined.

1. A breast implant (1) for women, comprising a flexible, but impermeable casing (2) and with a filling (3) located in the casing, the implant has an interior in which at least one electrical pole or one electrode (4) is located, and on an outside at least one additional electrical pole or one electrode (5) is provided, the two poles or electrodes are connected to an electrical resistance or impedance measurement device (6), and the filling (3) of the implant is electrically conductive.

2. The breast implant according to claim 1, wherein the two poles or electrodes (4, 5) are connected to a transponder (6) that contains the measurement device and is constructed for the transmission of measurement values to an external receiver (13, 14).

3. The breast implant according to claim 2, wherein the transponder (6) contains a resistor measurement bridge as the measurement device.

4. The breast implant according to claim 1, wherein the electrical pole or the electrode (4) located in the interior of the implant is arranged or fastened on an inner side to a rear wall or back side (7) of the implant as seen in a position of use.

5. The breast implant according to claim 1, wherein the pole or the electrode (5) located on the outside is arranged or fastened on an outer side to the rear wall or back side (7) of the implant as seen in the position of use.

6. The breast implant according to claim 1, wherein the two poles or electrodes (4, 5) are at least one of separated from each other electrically by the casing (2) of the implant or insulated relative to each other.

7. The breast implant according to claim 1, wherein at a position or on a wall holding the outer electrode or the outer pole (5), a recess for the pole or the electrode is provided and the pole or the electrode (5) is countersunk in the position of use relative to a surface of the outer side at least partially or such that the outer side of the pole or the electrode is flush with the outer side of the wall holding it.

8. The breast implant according to claim 2, wherein an opening (8) for introduction of the implant filling and a closure (9) for the opening are provided on a rear wall (7) of the implant and the outer pole or the electrode (5), the transponder (6), and the inner pole or the electrode (4) are arranged on or in the closure (9).

9. The breast implant according to claim 1, wherein lines or electrically conductive arms (10, 11, 12) extend from the pole or electrode and are arranged at least on a rear outer side of a back wall (7) of the implant in a position of use or extend beyond a front side of the implant and are connected to the outer pole or the electrode (5).

10. The breast implant according to claim 1, wherein additional, electrically conductive arms are constructed as elastically deformable, electrically conductive bands.
